(19) United States
(12) Patent Application Publication
(10) Pub. No.: US 2011/0009420 A1
(43) Pub. Date: Jan. 13, 2011

(54) Histamine H3 Receptor Ligands

(75) Inventor: Knud Erik Andersen, Brondby (DK)

Correspondence Address: High Point Pharmaceuticals, LLC, 4170 Mendenhall Oaks Parkway, High Point, NC, 27265, US

(73) Assignee: HIGH POINT PHARMACEUTICALS, LLC, High Point, NC (US)

(21) Appl. No.: 12/883,852

(22) Filed: Sep. 16, 2010

Related U.S. Application Data
(63) Continuation of application No. PCT/US09/40025, filed on Apr. 9, 2009.
(60) Provisional application No. 61/044,289, filed on Apr. 11, 2008.

(51) Int. Cl. A61K 31/496; C07D 241/04; A61K 31/495; C07D 401/14; A61P 25/28

(52) U.S. Cl. 514/253.11; 544/391; 514/255.01; 544/364

(57) ABSTRACT

The present invention provides compounds of the formula

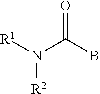

wherein R¹, R² and B are as herein described, pharmaceutical compositions comprising these compounds, use of these compounds for the preparation of pharmaceutical compositions and methods of use thereof for the treatment and/or prevention of disorders and diseases wherein modulation of the H₃ receptor is beneficial.

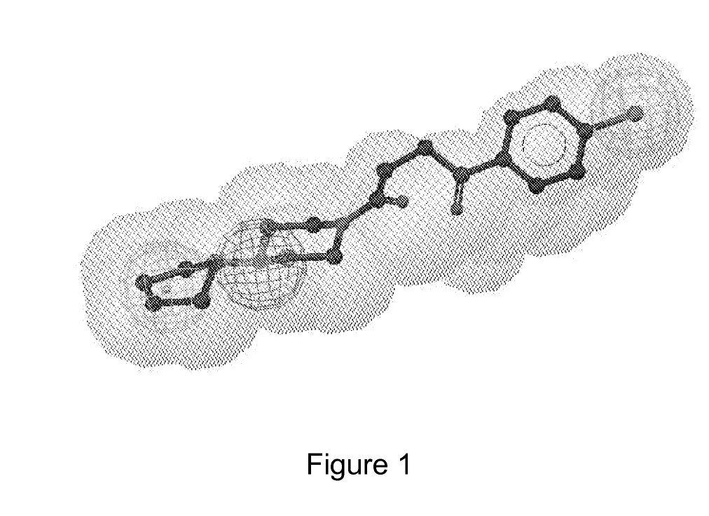

### BACKGROUND OF THE INVENTION

The present invention provides compounds, compositions comprising amide derivatives, including amido-piperazines, use of these amide derivatives for the preparation of pharmaceutical compositions and methods of use thereof for the treatment of disorders and diseases wherein modulation of the H₃ receptor is beneficial.

The histamine H₃ receptor, discovered in 1983, is a presynaptic autoreceptor mainly localized in the central nervous system and which, among other things, modulates the biosynthesis and release of histamine from histaminergic neurons. The human, rat, mouse, monkey, and guinea pig histamine H₃ receptors have been cloned, and this has led to renewed interest in histamine research. Because the histamine H₃ receptor is widely distributed in the CNS both as an autoreceptor on histaminergic neurons, but also as a presynaptic heteroreceptor on presynaptic terminals of other neurotransmitter systems, an important influence on physiological processes of centrally acting histamine H₃ receptor antagonists is anticipated. Centrally and peripherally administered histamine H₃ antagonists lead to increased central histamine levels, and preclinical rodent data support the idea that H₃ antagonists may be useful for the treatment of obesity in humans.

Previously several attempts to prepare H₃ receptor ligands containing one or more basic nitrogens have been described. Previously, the discovery of a series of 1-alkyl-4-acylpiperazines has been described that were ligands at the human histamine H₃ receptor. The most potent acylpiperazines found were those derived from 4-aryl-4-oxobutyric acid and 1-alkylpiperazines, illustrated by compound 1.

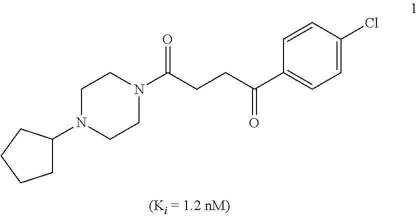

### SUMMARY OF THE INVENTION

The amine portion of compound 1 may be crucial for potency at the H₃ receptor. Therefore, to further investigate this part of the molecule, a two component virtual library 2 was designed around compound 1.

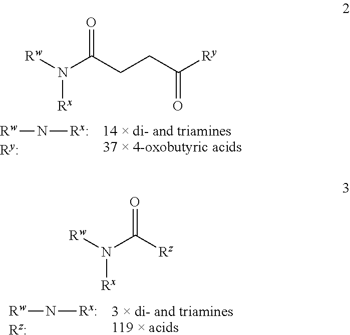

In this library, a series of the new di- and triamines were introduced, keeping the acid part relatively fixed with 4-oxobutyric acid derivatives. The virtual library was searched with a catalyst pharmacophore model generated from compound 1. The resulting compounds were prepared and tested to give several hits that identified 3 new di- and triamine moieties that were beneficial for potency at the H₃ receptor. Based on these 3 amines, a second library 3 was designed in which the acid part was varied considerably. Preparation and screening of library 3 resulted in several hits that identified one triamine and several acids as optimal for potency at the H₃ receptor. Finally, re-synthesis and testing of several of the hits identified from library 3, showed that they have high potency at the H₃ receptor. The synthesis and biological activity of these new potent H₃ receptor ligands will be described below.

### BRIEF DESCRIPTION OF DRAWING

The FIGURE is a drawing showing a pharmacophore model of compound 1 as used to develop compounds according to the present invention.

### DETAILED DESCRIPTION OF THE INVENTION

### Compounds

Embodiments of the present invention comprise substituted amide derivatives, compositions, and methods of use thereof. The present invention may be embodied in a variety of ways.

In a first aspect, the present invention provides amide derivatives which are modulators of H₃ which may be useful for the management and treatment of disease where modulation of the H₃ receptor is beneficial.

In another aspect, the present invention provides a compound of Formula (I):

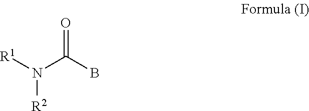

wherein
R¹ is selected from the group consisting of hydrogen, alkyl and —(CH₂)ₙR^{a}, wherein
   R^{a} is selected from the group consisting of haloalkyl, cycloalkyl, heterocyclyl, alkoxy, haloalkoxy, wherein the cycloalkyl and the heterocyclyl are substituted with from zero to four R^{d}; and
   n is an integer from 0 to 4, wherein
      R^{d} is selected from the group consisting of alkyl, halogen, hydroxyl, —(CH₂)ₘ-aryl, haloalkyl, —C(O)—R^{a′}, alkoxy and alkoxyalkyl, wherein
         m is an integer from 0 to 4;
         R^{a′} is selected from the group consisting of alkyl, -alkylene-cycloalkyl, and -alkylene-heterocyclyl; and
R² is hydrogen or —(CH₂)ᵣ(CHR^{e})ₒ(CH₂)ₙN(R^{b})(R^{c}), provided that when R¹ is hydrogen, R² is —(CH₂)ᵣ(CHR^{e})ₒ(CH₂)ᵣN(R^{b})(R^{c}), wherein
   R^{e} is selected from the group consisting of halogen, hydroxyl, alkyl, haloalkyl, alkoxy and alkoxyalkyl;
   r is an integer from 0 to 4;
   o is either 0 or 1;
   R^{b} and R^{c} are
      independently selected from the group consisting of alkyl, haloalkyl and alkyoxyalkyl; or
      are taken together with the nitrogen atom thereattached to form a heterocyclyl group, wherein the heterocyclyl group contains from zero to two additional ring heteroatoms and wherein the heterocyclyl group is substituted with one to four R^{f}, wherein
         R^{f} is selected from the group consisting of hydrogen, alkyl, haloalkyl and alkyoxyalkyl;
or
R¹ and R² are taken together with the nitrogen atom thereattached to form a heterocyclyl group, wherein the heterocyclyl group contains from zero to two additional ring heteroatoms and wherein the heterocyclyl group is substituted with one to four Rⁱ, wherein
   Rⁱ is selected from the group consisting of alkyl, halogen, haloalkyl, cycloalkyl and —(CH₂)ₙ-heterocyclyl;
B is selected from the group consisting of —(CH₂)ₚ-aryl, -alkenylene-aryl, —(CH₂)ₚ-heteroaryl, -alkenylene-heteroaryl, —(CH₂)_{q}—C(O)—(CH₂)ₚ-aryl, and —(CH₂)_{q}—C(O)—(CH₂)ₚ-heteroaryl, —(CH₂)ₚ-fused cycloalkylaryl and —(CH₂)_{q}—C(O)—(CH₂)ₚ-fused cycloalkylaryl wherein the aryl and heteroaryl groups each independently are subsubstituted with from zero to four R^{g}, wherein
   p is an integer from 0 to four;
   q is an integer from 0 to four; and
   each R^{g} independently is selected from the group consisting of hydrogen, —O—Ar¹,
      Ar¹, alkyl, alkoxy, hydroxyl, halogen, haloalkyl and alkoxyalkyl, wherein the
      Ar¹ is substituted with from zero to four R^{h}, wherein
         Ar1 is aryl or heteroaryl;
         R^{h} is selected from the group consisting of hydrogen, alkyl, alkoxy, hydroxyl, halogen, haloalkyl and alkoxyalkyl;
            or a pharmaceutically acceptable salt salt thereof.

In one embodiment, R¹ and R² form a piperazine ring substituted by at least one Rⁱ.

In one embodiment, the compound of Formula (I) is represented by Formula (I-A):

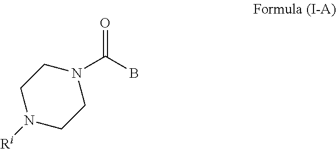

wherein the variables are as defined above,
or a pharmaceutically acceptable salt thereof.

In another embodiment, the compound of Formula (I) is represented by Formula (I-B):

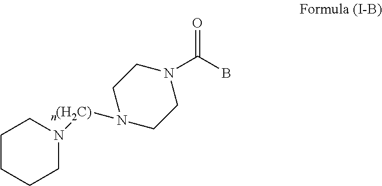

Pharmaceutically-acceptable salts of the compounds of the present invention, where a basic or acidic group is present in the structure, are also included within the scope of the invention. The term “pharmaceutically acceptable salts” refers to non-toxic salts of the compounds of this invention which are generally prepared by reacting the free base with a suitable organic or inorganic acid or by reacting the acid with a suitable organic or inorganic base. Representative salts include the following salts: Acetate, Benzenesulfonate, Benzoate, Bicarbonate, Bisulfate, Bitartrate, Borate, Bromide, Calcium Edetate, Camsylate, Carbonate, Chloride, Clavulanate, Citrate, Dihydrochloride, Edetate, Edisylate, Estolate, Esylate, Fumarate, Gluceptate, Gluconate, Glutamate, Glycollylarsanilate, Hexylresorcinate, Hydrabamine, Hydrobromide, Hydrocloride, Hydroxynaphthoate, Iodide, Isethionate, Lactate, Lactobionate, Laurate, Malate, Maleate, Mandelate, Mesylate, Methylbromide, Methylnitrate, Methylsulfate, Monopotassium Maleate, Mucate, Napsylate, Nitrate, N-methylglucamine, Oxalate, Pamoate (Embonate), Palmitate, Pantothenate, Phosphate/diphosphate, Polygalacturonate, Potassium, Salicylate, Sodium, Stearate, Subacetate, Succinate, Tannate, Tartrate, Teoclate, Tosylate, Triethiodide, Trimethylammonium and Valerate. When an acidic substituent is present, such as—COOH, there can be formed the ammonium, morpholinium, sodium, potassium, barium, calcium salt, and the like, for use as the dosage form. Also intended as pharmaceutically acceptable acid addition salts are the hydrates which the present compounds are able to form. The acid addition salts may be obtained as the direct products of compound synthesis. In the alternative, the free base may be dissolved in a suitable solvent containing the appropriate acid, and the salt isolated by evaporating the solvent or otherwise separating the salt and solvent. When a basic group is present, such as amino or a basic heteroaryl radical, such as pyridyl, an acidic salt, such as hydrochloride, hydrobromide, phosphate, sulfate, trifluoroacetate, trichloroacetate, acetate, oxlate, maleate, pyruvate, malonate, succinate, citrate, tartarate, fumarate, mandelate, benzoate, cinnamate, methanesulfonate, ethanesulfonate, picrate and the like.

Other salts which are not pharmaceutically acceptable may be useful in the preparation of compounds of the invention and these form a further aspect of the invention.

Thus, in a further embodiment, there is provided a pharmaceutical composition comprising a compound of the present invention, or a pharmaceutically acceptable salt thereof, and a pharmaceutically acceptable carrier, excipient, diluent, or mixture thereof.

Embodiments of the invention described herein are additionally directed to pharmaceutical compositions and use thereof in methods for modulating the H₃ receptor, which methods comprise administering to a subject in need of modulation of the H₃ receptor a compound of Formula (I), defined above, or a pharmaceutically acceptable salt thereof.

### DEFINITIONS

As used herein, the terms “disease”, “condition” and “disorder” as used herein are used interchangeably to specify a state of a patient which is not the normal physiological state of man.

As used herein, the term “medicament” as used herein means a pharmaceutical composition suitable for administration of the pharmaceutically active compound to a patient.

As used herein, the term “pharmaceutically acceptable” as used herein means suited for normal pharmaceutical applications, i.e. giving rise to no adverse events in patients etc.

As used herein, the term “effective amount” as used herein means a dosage which is sufficient in order for the treatment of the patient to be effective compared with no treatment.

As used herein, the term “therapeutically effective amount” of a compound as used herein means an amount sufficient to alleviate or partially arrest the clinical manifestations of a given disease and its complications. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary.

As used herein, the term “lower” refers to a group having between one and six carbons.

As used herein, the term “alkyl” refers to a straight or branched chain hydrocarbon having from one to ten carbon atoms, optionally substituted and multiple degrees of substitution being allowed. Examples of “alkyl” as used herein include, but are not limited to, methyl, n-butyl, t-butyl, n-pentyl, isobutyl and isopropyl. As used herein, the term “haloalkyl” refers to a straight or branched chain hydrocarbon having from one to ten carbon atoms, substituted with at least one halogen atom and optionally substituted at the remaining positions with a halogen atom. A haloalkyl group may be substituted with one or more types of halogen atoms. Examples of “haloalkyl” as used herein include, but are not limited to, a trifluoromethyl group and a 2,2,2-trifluoroethyl group.

As used herein, the term “perhaloalkyl” refers to a straight or branched chain hydrocarbon having from one to ten carbon atoms, where each position for sustitution is substituted with a halogen atom. A perhaloalkyl group may be substituted with one or more types of halogen atoms. Examples of “perhaloalkyl” as used herein include, but are not limited to, a trifluoromethyl group and a 1,1-dichloro-2,2,2-trifluoroethyl group.

As used herein, the term “alkylene” refers to a straight or branched chain divalent hydrocarbon radical having from one to ten carbon atoms, optionally substituted and multiple degrees of substitution being allowed. Examples of “alkylene” as used herein include, but are not limited to, methylene, ethylene and 2-methyl-propylene. As used herein, the term “alkyline” refers to a straight or branched chain trivalent hydrocarbon radical having from one to ten carbon atoms, optionally substituted and multiple degrees of substitution being allowed. Examples of “alkyline” as used herein include, but are not limited to, methane and ethyline.

As used herein, the term “alkenyl” refers to a hydrocarbon radical having from two to ten carbons and at least one carbon-carbon double bond, optionally substituted and multiple degrees of substitution being allowed. Examples of “alkenyl” as used herein include, but are not limited to, 3,3-dimethyl-but-1-enyl and 4-hex-1-enyl.

As used herein, the term “alkenylene” refers to a straight or branched chain divalent hydrocarbon radical having from two to ten carbon atoms and one or more carbon-carbon double bonds, optionally substituted and multiple degrees of substitution being allowed. Examples of “alkenylene” as used herein include, but are not limited to, ethene-1,2-diyl, propene-1,3-diyl and methylene-1,1-diyl.

As used herein, the term “alkynyl” refers to a hydrocarbon radical having from two to ten carbons and at least one carbon-carbon triple bond, optionally substituted and multiple degrees of substitution being allowed. Examples of “alkynyl” as used herein include, but are not limited to, 4-hex-1ynyl and 3,3-dimethyl-but-1ynyl.

As used herein, the term “alkynylene” refers to a straight or branched chain divalent hydrocarbon radical having from two to ten carbon atoms and one or more carbon-carbon triple bonds, optionally substituted and multiple degrees of substitution being allowed. Examples of “alkynylene” as used herein include, but are not limited to, ethyne-1,2-diyl and propyne-1,3-diyl.

As used herein, the terms “haloaliphatic”, “haloalkyl”, “haloalkenyl” and “haloalkoxy” refer to an aliphatic, alkyl, alkenyl or alkoxy group, as the case may be, substituted with one or more halogen atoms.

As used herein, “cycloalkyl” refers to a non-aromatic alicyclic hydrocarbon group and optionally possessing one or more degrees of unsaturation, having from three to twelve carbon atoms, optionally substituted and multiple degrees of substitution being allowed. Examples of “cycloalkyl” as used herein include, but are not limited to, cyclopropyl, cyclobutyl, cyclopentyl, cyclohexyl, cycloheptyl, cyclooctyl and adamantyl.

As used herein, the term “cycloalkylene” refers to an non-aromatic alicyclic divalent hydrocarbon radical having from three to twelve carbon atoms and optionally possessing one or more degrees of unsaturation, optionally substituted with substituents and multiple degrees of substitution being allowed. Examples of “cycloalkylene” as used herein include, but are not limited to, cyclopropyl-1,1-diyl, cyclopropyl-1,2-diyl, cyclobutyl-1,2-diyl, cyclopentyl-1,3-diyl, cyclohexyl-1,4-diyl, cycloheptyl-1,4-diyl and cyclooctyl-1,5-diyl.

As used herein, the term “heterocyclic” or the term “heterocyclyl” refers to a non-aromatic three to twelve-membered heterocyclic ring optionally possessing one or more degrees of unsaturation, containing one or more heteroatomic substitutions selected from S, SO, SO₂, O, or N, optionally substituted and multiple degrees of substitution being allowed. Such a ring may be optionally fused to from one to three of another “heterocyclic” ring(s) or cycloalkyl ring(s). Examples of “heterocyclyl” include, but are not limited to, tetrahydrofuran, 1,4-dioxane, 1,3-dioxane, piperidine, pyrrolidine, morpholine and piperazine.

As used herein, the term “heterocyclylene” refers to a non-aromatic three to twelve-membered heterocyclic ring diradical optionally having one or more degrees of unsaturation containing one or more heteroatoms selected from S, SO, SO₂, O, or N, optionally substituted and multiple degrees of substitution being allowed. Such a ring may be optionally fused to from one to three benzene rings or to one to three of another “heterocyclic” rings or cycloalkyl rings. Examples of “heterocyclylene” include, but are not limited to, tetrahydrofuran-2,5-diyl, morpholine-2,3-diyl, pyran-2,4-diyl, 1,4-dioxane-2,3-diyl, 1,3-dioxane-2,4-diyl, piperidine-2,4-diyl, piperidine-1,4-diyl, pyrrolidine-1,3-diyl, morpholine-2,4-diyl and piperazine-1,4-diyl.

As used herein, the term “aryl” refers to a benzene ring or to benzene ring fused to one to three benzene rings, optionally substituted and multiple degrees of substitution being allowed. Examples of aryl include, but are not limited to, phenyl, 2-naphthyl, 1-naphthyl and 1-anthracenyl.

As used herein, the term “arylene” refers to a benzene ring diradical or to a benzene ring system diradical fused to one to three optionally substituted benzene rings, optionally substituted and multiple degrees of substitution being allowed. Examples of “arylene” include, but are not limited to, benzene-1,4-diyl and naphthalene-1,8-diyl.

As used herein, the term “heteroaryl” refers to a five- to seven-membered aromatic ring, or to a polycyclic (up to three rings) aromatic ring, containing one or more nitrogen, oxygen, or sulfur heteroatoms, where N-oxides and sulfur monoxides and sulfur dioxides are permissible heteroaromatic substitutions, optionally substituted and multiple degrees of substitution being allowed. For polycyclic heteroaryl aromatic ring systems, one or more of the rings may contain one or more heteroatoms. Examples of “heteroaryl” used herein include, but are not limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, quinazoline, benzofuran, benzothiophene, indole, and indazole.

As used herein, the term “heteroarylene” refers to a five- to seven-membered aromatic ring diradical, or to a polycyclic (up to three rings) heterocyclic aromatic ring diradical, containing one or more nitrogen, oxygen, or sulfur heteroatoms, where N-oxides and sulfur monoxides and sulfur dioxides are permissible heteroaromatic substitutions, optionally substituted and multiple degrees of substitution being allowed. For polycyclic aromatic ring system diradicals, one or more of the rings may contain one or more heteroatoms. Examples of “heteroarylene” used herein include, but are not limited to, furan-2,5-diyl, thiophene-2,4-diyl, 1,3,4-oxadiazole-2,5-diyl, 1,3,4-thiadiazole-2,5-diyl, 1,3-thiazole-2,4-diyl, 1,3-thiazole-2,5-diyl, pyridine-2,4-diyl, pyridine-2,3-diyl, pyridine-2,5-diyl, pyrimidine-2,4-diyl and quinoline-2,3-diyl.

As used herein, the term “fused cycloalkylaryl” refers to one or two cycloalkyl groups fused to an aryl group, the aryl and cycloalkyl groups having two atoms in common, and wherein the aryl group is the point of substitution. Examples of “fused cycloalkylaryl” used herein include 5-indanyl, 5,6,7,8-tetrahydro-2-naphthyl and

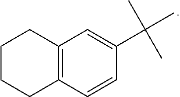

As used herein, the term “direct bond”, where part of a structural variable specification, refers to the direct joining of the substituents flanking (preceding and succeeding) the variable taken as a “direct bond”. Where two or more consecutive variables are specified each as a “direct bond”, those substituents flanking (preceding and succeeding) those two or more consecutive specified “direct bonds” are directly joined.

As used herein, the term “alkoxy” refers to the group RₓO—, where Rₓ is alkyl.

As used herein, the term “alkenyloxy” refers to the group RₓO—, where Rₓ is alkenyl.

As used herein, the term “alkynyloxy” refers to the group RₓO—, where Rₓ is alkynyl.

As used herein, the term “alkylsulfanyl” refers to the group RₓS—, where Rₓ is alkyl.

As used herein, the term “alkenylsulfanyl” refers to the group RₓS—, where Rₓ is alkenyl.

As used herein, the term “alkynylsulfanyl” refers to the group RₓS—, where Rₓ is alkynyl.

As used herein, the term “alkylsulfinyl” refers to the group RₓS(O)—, where Rₓ is alkyl.

As used herein, the term “alkenylsulfinyl” refers to the group RₓS(O)—, where Rₓ is alkenyl.

As used herein, the term “alkynylsulfinyl” refers to the group RₓS(O)—, where Rₓ is alkynyl.

As used herein, the term “alkylsulfonyl” refers to the group RₓSO₂—, where Rₓ is alkyl.

As used herein, the term “alkenylsulfonyl” refers to the group RₓSO₂—, where Rₓ is alkenyl.

As used herein, the term “alkynylsulfonyl” refers to the group RₓSO₂—, where Rₓ is alkynyl.

As used herein, the term “acyl” refers to the group RₓC(O)—, where Rₓ is alkyl, alkenyl, alkynyl, cycloalkyl, cycloalkenyl, or heterocyclyl.

As used herein, the term “aroyl” refers to the group RₓC(O)—, where Rₓ is aryl.

As used herein, the term “heteroaroyl” refers to the group RₓC(O)—, where Rₓ is heteroaryl.

As used herein, the term “alkoxycarbonyl” refers to the group RₓOC(O)—, where Rₓ is alkyl.

As used herein, the term “acyloxy” refers to the group RₓC(O)O—, where Rₓ is alkyl, alkenyl, alkynyl, cycloalkyl, cycloalkenyl, or heterocyclyl.

As used herein, the term “aroyloxy” refers to the group RₓC(O)O—, where Rₓ is aryl.

As used herein, the term “heteroaroyloxy” refers to the group RₓC(O)O—, where Rₓ is heteroaryl.

As used herein, the term “optionally” means that the subsequently described event(s) may or may not occur, and includes both event(s) which occur and events that do not occur.

As used herein, the term “substituted” refers to substitution of one or more hydrogens of the designated moiety with the named substituent or substituents, multiple degrees of substitution being allowed unless otherwise stated, provided that the substitution results in a stable or chemically feasible compound. A stable compound or chemically feasible compound is one in which the chemical structure is not substantially altered when kept at a temperature from about −80° C. to about +40° C., in the absence of moisture or other chemically reactive conditions, for at least a week, or a compound which maintains its integrity long enough to be useful for therapeutic or prophylactic administration to a patient. The phrase “one or more substituents”, as used herein, refers to a number of substituents that equals from one to the maximum number of substituents possible based on the number of available bonding sites, provided that the above conditions of stability and chemical feasibility are met.

As used herein, the terms “contain” or “containing” can refer to in-line substitutions at any position along the above defined alkyl, alkenyl, alkynyl or cycloalkyl substituents with one or more of any of O, S, SO, SO₂, N, or N-alkyl, including, for example, —CH₂—O—CH₂—, —CH₂—SO₂—CH₂—, —CH₂—NH—CH₃ and so forth.

Whenever the terms “alkyl” or “aryl” or either of their prefix roots appear in a name of a substituent (e.g. arylalkoxyaryloxy) they shall be interpreted as including those limitations given above for “alkyl” and “aryl”. Designated numbers of carbon atoms (e.g. C₁₋₁₀) shall refer independently to the number of carbon atoms in an alkyl, alkenyl or alkynyl or cyclic alkyl moiety or to the alkyl portion of a larger substituent in which the term “alkyl” appears as its prefix root.

As used herein, the term “oxo” shall refer to the substituent ═O.

As used herein, the term “halogen” or “halo” refers iodine, bromine, chlorine or fluorine.

As used herein, the term “mercapto” refers to the substituent —SH.

As used herein, the term “carboxy” refers to the substituent —COOH.

As used herein, the term “cyano” refers to the substituent —CN.

As used herein, the term “aminosulfonyl” refers to the substituent —SO₂NH₂.

As used herein, the term “carbamoyl” refers to the substituent —C(O)NH₂.

As used herein, the term “sulfanyl” refers to the substituent —S—.

As used herein, the term “sulfinyl” refers to the substituent —S(O)—.

As used herein, the term “sulfonyl” refers to the substituent —S(O)₂—.

As used herein the terms “pharmaceutically acceptable carrier”, “pharmaceutically acceptable diluent”, and pharmaceutically acceptable excipient” means the carrier, diluent or excipient must be compatible with the other ingredients of the formulation and not deleterious to the recipient thereof.

The compounds can be prepared according to the following reaction Schemes (in which variables are as defined before or are defined) using readily available starting materials, and reagents. In these reactions, it is also possible to make use of variants which are themselves known to those of ordinary skill in this art, but are not mentioned in greater detail.

### Methods

A pharmacophore model for compound 1 was generated by conformational analysis in Maestro version 7 using the MMFF force field, water as the solvation model and the MCMM method for the conformational search. The pharmacophore features were mapped in Catalyst version 4.6 to finally give the pharmacophore model presented in the FIGURE. A Catalyst database from the virtual scope of library 2 was built with the fast generation method and with a maximum of 100 conformations. This database was searched with the Fast Flexible Search method to select a subset of compounds for synthesis.

The synthetic strategy for libraries 2 and 3 was based on the previously reported solid phase synthesis of amides from the 4-hydroxy-2,3,5,6-tetrafluorobenzamido (TFP) resin 4 (Scheme 1).

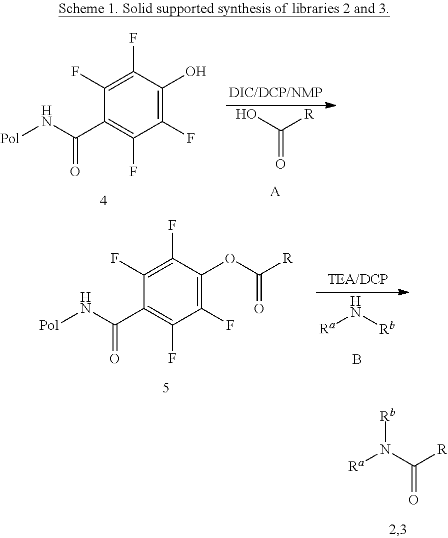

This method was well suited for automated parallel synthesis of the two libraries 2 and 3 on an Advanced ChemTech 384 HTS synthesizer. The TFP resin (0.045 mmole scale) was dispensed to each well of the synthesizer and the appropriate acid in excess was coupled to the resin using DIC in DCP/NMP to give the resin-bound intermediate esters 5. The appropriate amine was then added as the limiting reagent (0.9 eq) in DCP in the presence of TEA to cleave the product from the solid support by nucleophilic attack. This afforded after evaporation of the volatiles the desired amides of library 2 and 3. Library 2 was screened for hits without any further purification, whereas library 3 was purified by automated HPLC-MS and quantified by ELS before screening.

Hits identified within the second library 3 were re-synthesised in solution and the potencies of the hit compounds were reconfirmed in the functional [³⁵S]GTPγS binding assay. Re-synthesis of these hits was accomplished through a standard amide coupling reaction in solution. The appropriate carboxylic acid was activated with HOAt and EDAC in DCM. The activated acid was then treated with the appropriate amine to give the desired amide derivatives that were isolated as their dihydrochloride salts (Table 3). The design of library 2 included 14 di- and triamines (B1-B14) which are featured below.

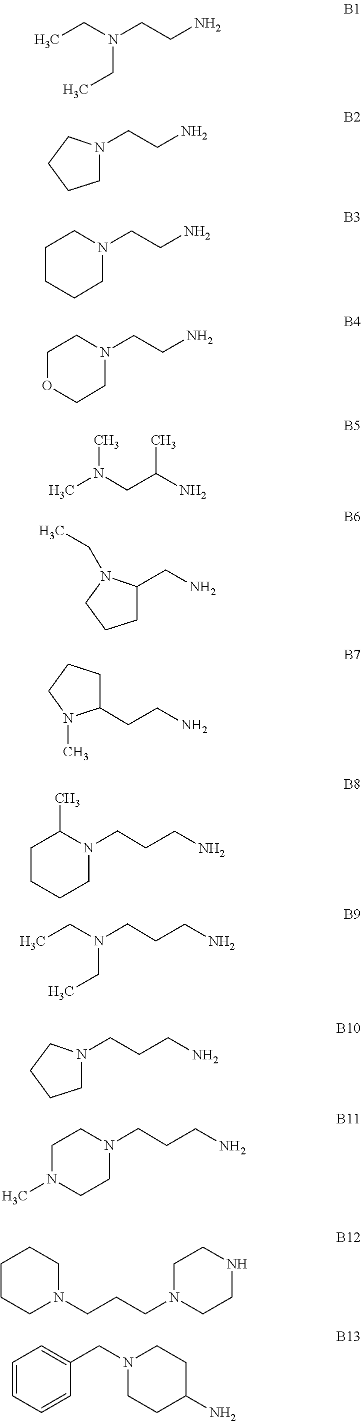

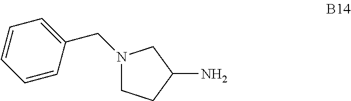

The acid building blocks from hit compounds identified within library 2 and 3 are shown below.

Library 2:

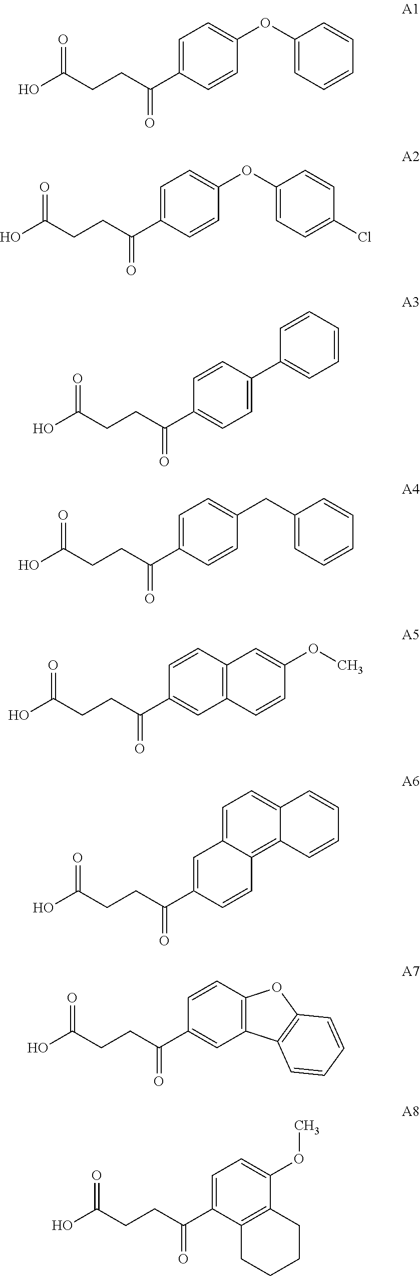

Library 3:

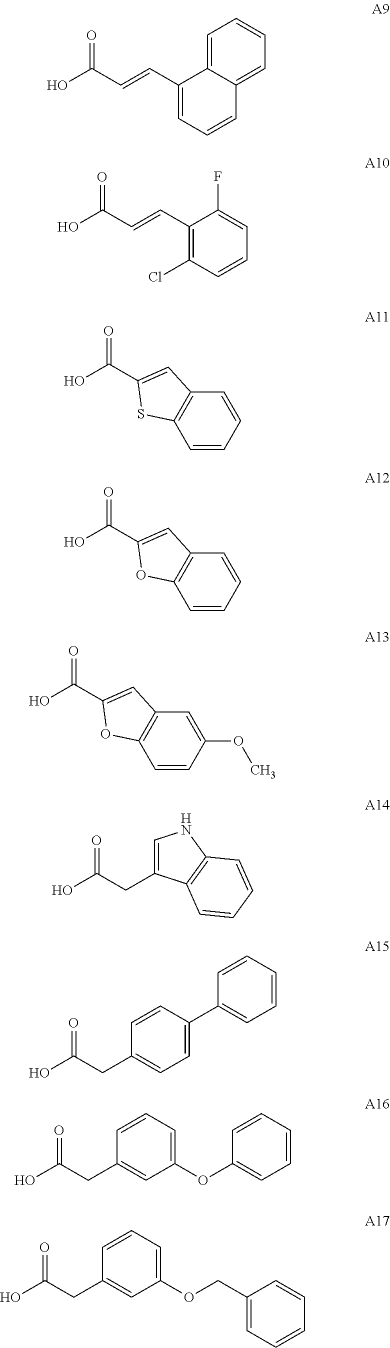

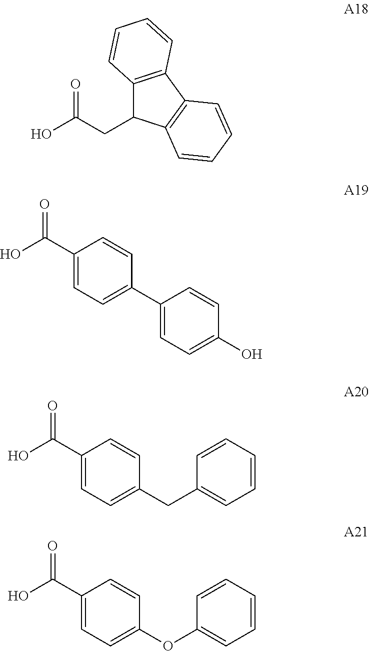

The 4-oxobutyric acid substructure of compound 1 was preserved in the design of library 2. In total 37 analogues of 4-oxobutyric acid were used in the design and the acid building blocks within the identified hit compounds from library 2 are shown above (A1-A8). From the total scope of 518 virtual compounds, 384 compounds were selected by the Catalyst pharmacophore model developed from compound 1 and the selected compounds were prepared by automated parallel synthesis as described above. The non-purified samples from library 2 were submitted for screening in the automated functional [³⁵S]GTPγS binding assay at an estimated final compound concentration of 1.4 μM. Purification was omitted to save time and resources for this hit-finding library. An exemplary sixteen hits inhibiting more than 40% of the GTPγS binding at this concentration are presented in Table 1.

TABLE 1
Compounds from library 2 inhibiting [³⁵S]GTPγS binding
more than 40% at 1.4 μM.

| | | | | | Inhibition of |
|---|---|---|---|---|---|
| | | [M + H]⁺ | [M + H]⁺ | Purity^{a} | [³⁵S]GTPγS^{b} |
| Entry | Compound | Calcd | Found | (%) | (%) |
| 1 | A1B8 | 409.5 | 409.4 | 71.9 | 41 |
| 2 | A1B10 | 381.5 | 381.2 | 64.9 | 51 |
| 3 | A1B12 | 464.6 | 464.2 | 64.7 | 57 |
| 4 | A2B8 | 444.0 | 443.2 | 73.2 | 54 |
| 5 | A2B10 | 415.9 | 415.1 | 69.2 | 59 |
| 6 | A2B12 | 499.1 | 498.2 | 66.8 | 57 |
| 7 | A3B8 | 393.5 | 393.3 | 63.1 | 45 |
| 8 | A3B10 | 365.5 | 365.1 | 54.7 | 44 |
| 9 | A3B12 | 448.6 | 448.2 | 64.4 | 46 |
| 10 | A4B10 | 379.5 | 379.2 | 62.0 | 42 |
| 11 | A4B12 | 462.7 | 462.2 | 62.8 | 40 |
| 12 | A5B10 | 369.5 | 369.2 | 62.6 | 46 |
| 13 | A5B12 | 452.6 | 452.4 | 69.6 | 52 |
| 14 | A6B12 | 472.6 | 472.2 | 77.9 | 51 |
| 15 | A7B12 | 462.6 | 462.2 | 62.4 | 42 |
| 16 | A8B7 | 373.5 | 373.4 | 78.6 | 41 |

^{a}LC/MS analysis as described in Experimental Section.
^{b}[³⁵S]GTPγS binding assay as described in Experimental Section at a final compound concentration of 1.4 μM.

The results outlined in Table 1 clearly put focus on the 3 amines B8, B10 and B12 as the amines with the greatest potential in a more focused library. In addition, these 3 amines appear in the hit compounds together with a limited number of carboxylic acids A1-A7, and frequently with the same carboxylic acid for more than one of the 3 amines. This should confirm the presence and validity of the hits identified.

Based on the 3 amines B8, B10 and B12 the design of library 3 was made to explore the acid part with 119 carboxylic acids. This resulted in a virtual scope of 357 compounds, which were all prepared by automated parallel synthesis. The 357 synthesised samples were submitted for automated LC-MS purification and quantification to recover 298 samples. The remaining 59 samples which could not be re-covered, represent 3 acids that were excluded from synthesis due to solubility problems and 28 acids that failed during either synthesis or purification. Generally, the 3 amines used in the design worked well. The 298 recovered and quantified samples were then screened in the automated functional [³⁵S]GTPγS binding assay at a final compound concentration of 3.5 μM and the 15 hits inhibiting more than 50% at this concentration are presented in Table 2.

TABLE 2
Compounds from library 3 inhibiting [³⁵S]GTPγS binding
more than 50% at 3.5 μM.

| | | [M + H]⁺ | [M + H]⁺ | Purity^{a,b} | Yield^{b} | Inhibition of |
|---|---|---|---|---|---|---|
| Entry | Compound | Calcd | Found | (%) | mg (%) | [³⁵S]GTPγS^{c} (%) |
| 1 | A3B10 | 365.5 | 365.1 | >99 | 8.5 (52) | 61 |
| 2 | A3B12 | 448.6 | 448.3 | >99 | 10.1 (50) | 59 |
| 3 | A9B12 | 392.6 | 392.2 | 97.5 | 5.2 (30) | 52 |
| 4 | A10B12 | 394.9 | 394.2 | 96.8 | 9.1 (51) | 59 |
| 5 | A11B12 | 372.5 | 372.4 | >99 | 6.9 (41) | 57 |
| 6 | A12B12 | 356.5 | 356.2 | >99 | 12.7 (79) | 70 |
| 7 | A13B12 | 386.5 | 386.2 | >99 | 9.7 (56) | 60 |
| 8 | A14B12 | 369.5 | 369.2 | 91.7 | 5.0 (30) | 73 |
| 9 | A15B12 | 406.6 | 406.4 | 98.6 | 11.7 (64) | 56 |
| 10 | A16B12 | 422.6 | 422.2 | 96.8 | 9.6 (51) | 50 |
| 11 | A17B12 | 436.6 | 436.2 | 97.9 | 11.4 (58) | 54 |
| 12 | A18B12 | 418.6 | 418.2 | 88.0 | 12.6 (67) | 74 |
| 13 | A19B12 | 408.6 | 408.3 | 95.4 | 2.5 (14) | 51 |
| 14 | A20B12 | 406.6 | 406.3 | 98.8 | 8.4 (46) | 53 |
| 15 | A21B12 | 408.6 | 408.1 | >99 | 11.5 (61) | 62 |

^{a}LC/MS analysis as described in Experimental Section.
^{b}Determined after automated purification and quantification as described in Experimental Section.
^{c}[³⁵S]GTPγS binding assay as described in Experimental Section at a final compound concentration of 3.5 μM.

The result of library 3 identifies the amine B12 as an exemplary building block as it is present in the major part of the most potent hit compounds. The acid part of the hit compounds generally seem to be more constrained than in the reference compound 1. Seven of the most potent hit compounds from library 3 were re-synthesized on a larger scale in solution and tested, with full dose-response curves, in the [³⁵S]GTPγS binding assay. The results are shown in Table 3.

TABLE 3
Certain hit compounds re-synthesized.

| | | | | | [³⁵S]GTPγS |
|---|---|---|---|---|---|
| | | | | | binding |
| No. | Structure | Mp.° C. | Formula | Anal^{b} | Kᵢ(nM) |
| 1 | 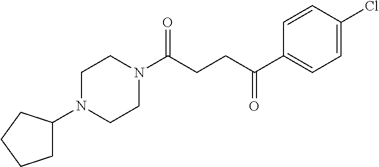 | | | | 2.2 |
| A11B12 | 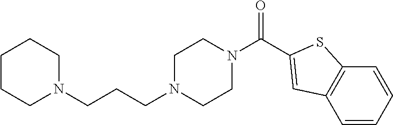 | 265-267^{a} | C₂₁H₂₉N₃OS, 2HCl, 2H₂O | C, H, N | 35 |
| A12B12 | 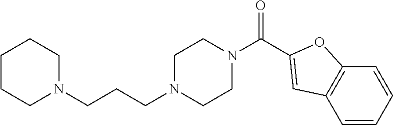 | 253-255^{a} | C₂₁H₂₉N₃O₂, 2HCl, 1.5H₂O | C, H, N | 19 |
| A13B12 | 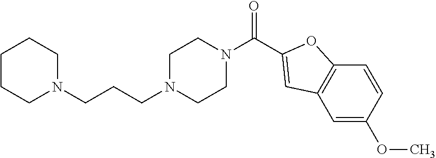 | 255-257^{a} | C₂₂H₃₁N₃O₃, 2HCl, 2H₂O | C, H, N | 16 |
| A14B12 | 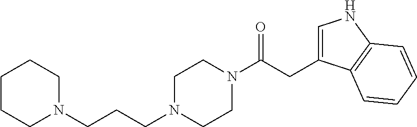 | 256-258^{a} | C₂₂H₃₂N₄O, 2HCl | C, H, N | 10 |
| A15B12 | 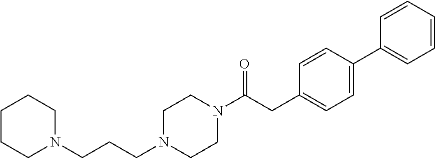 | 274-276^{a} | C₂₆H₃₅N₃O, 2HCl, H₂O | C, H, N | 51 |
| A18B12 | 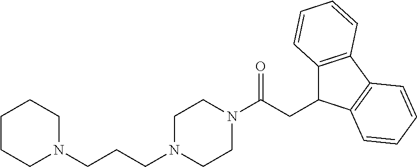 | 263-265^{a} | C₂₇H₃₅N₃O, 2HCl, H₂O | C, H, N^{c} | 7.4 |
| A21B12 | 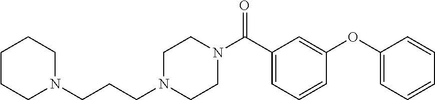 | 274-276^{a} | C₂₅H₃₃N₃O₂, 2HCl | C, H, N | 23 |

Crystallized from ^{a}CH₃CN;
^{b}Microanalytical data found were within ±0.4 % of the calculated value for the formula in the previous column, unless otherwise indicated.
^{c}C: Calc: 63.77; Found: 63.28.

As can be seen from Table 3, the potencies of the hit compounds were reconfirmed in the functional [³⁵S]GTPγS binding assay. Furthermore, some of the hit compounds (i.e. A14B12, A18B12) have potencies in the low nanomolar range, which is comparable with the potency of compound 1. This new series of lead structures may now be further optimised and evaluated for their biological potential.

Table 4 below shows the microanalytical data obtained for the compounds of Table 3.

TABLE 4
Microanalytical data of compounds of the present invention.

| | | Microanalysis Calc.(Found) | | |
|---|---|---|---|---|
| No. | Formula | C | H | N |
| A11B12 | C₂₁H₂₉N₃OS, 2HCl, 2H₂O | 52.49 | 7.34 | 8.75 |
| | | (52.74) | (7.28) | (8.71) |
| A12B12 | C₂₁H₂₉N₃O₂, 2HCl, 1.5H₂O | 55.38 | 7.52 | 9.23 |
| | | (55.42) | (7.35) | (9.09) |
| A13B12 | C₂₂H₃₁N₃O₃, 2HCl, H₂O | 55.46 | 7.40 | 8.82 |
| | | (55.42) | (7.37) | (8.74) |
| A14B12 | C₂₂H₃₂N₄O, 2HCl | 59.86 | 7.76 | 12.69 |
| | | (59.58) | (7.71) | (12.50) |
| A15B12 | C₂₆H₃₅N₃O, 2HCl, H₂O | 62.90 | 7.92 | 8.46 |
| | | (63.04) | (7.90) | (8.50) |
| A18B12 | C₂₇H₃₅N₃O, 2HCl, H₂O | 63.77 | 7.73 | 8.26 |
| | | (63.28) | (7.61) | (8.18) |
| A21B12 | C₂₅H₃₃N₃O₂, 2HCl | 62.50 | 7.34 | 8.75 |
| | | (62.27) | (7.32) | (8.57) |

The present invention includes compounds formed from the combination of any of B1 through B14 with any of A1 through A21. The point of attachment for the B group of compounds (B1 through B14) and the A group of compounds (A1 through A21) is at the * shown in each compound.

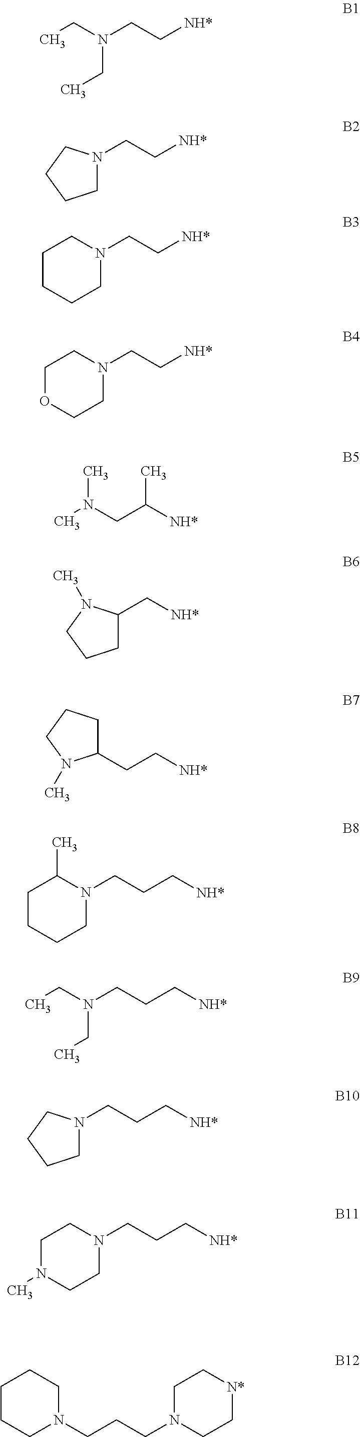

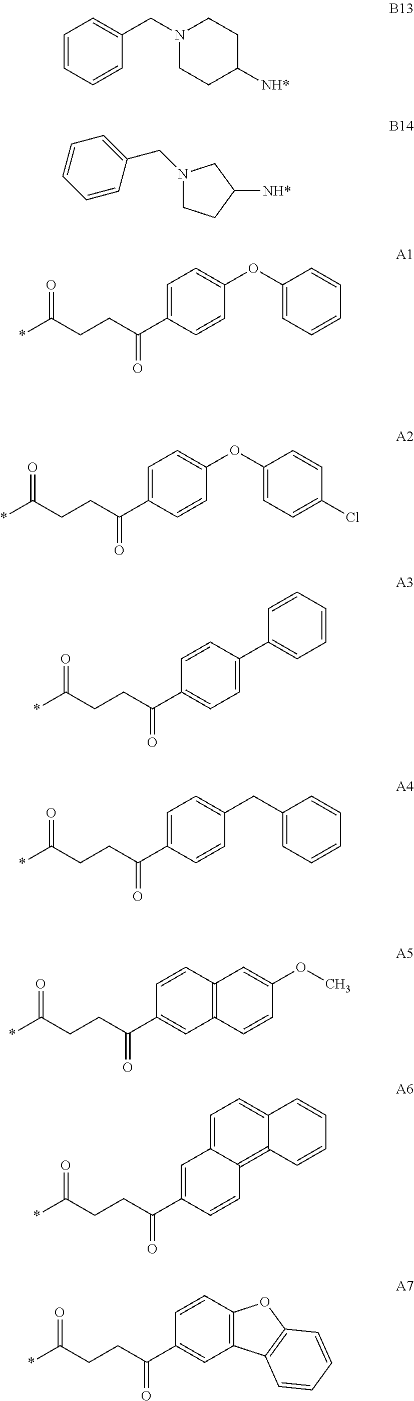

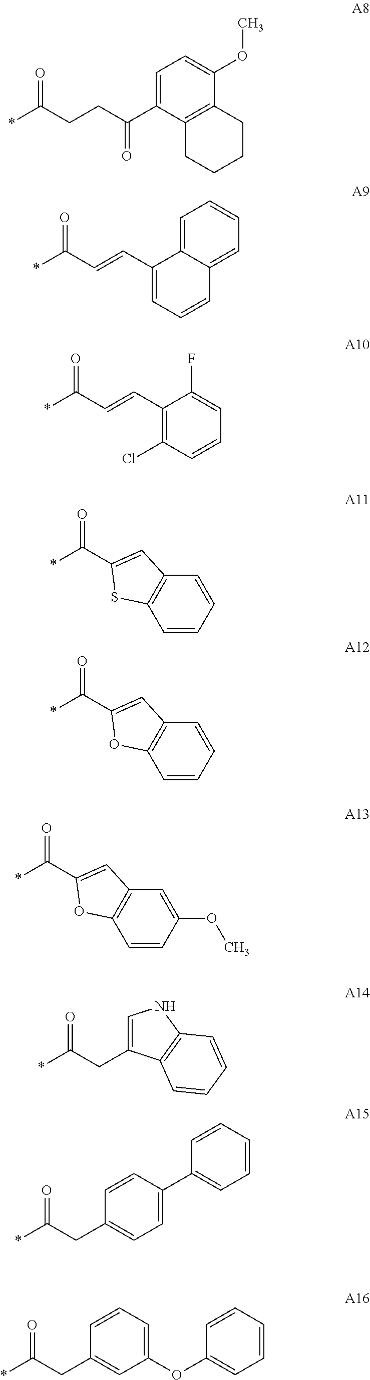

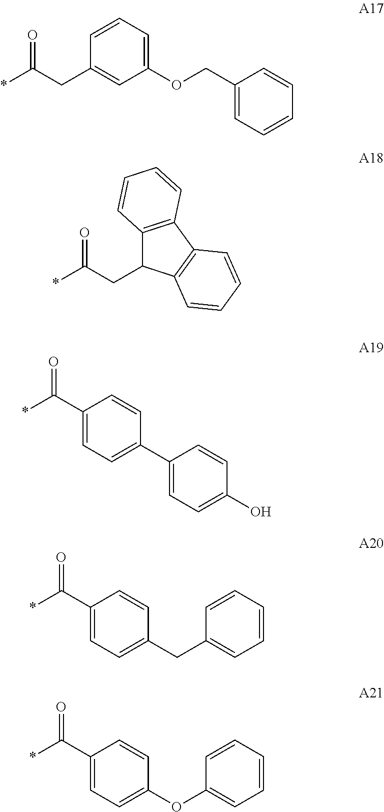

In particular, the present invention may include compounds wherein any of B7, B8, B10 and B12 are coupled to any of A1 through A8. In aother embodiment, the present invention may include compounds wherein B12 is coupled to any of A9 through A21.

### Methods of Medical Treatment

Due to their interaction with the histamine H3 receptor, the compounds of this invention as defined in the claims below and elsewhere in this specification are useful in the treatment of a wide range of conditions and disorders in which an interaction with the histamine H₃ receptor is beneficial. Thus, the compounds may find use, for example, in the treatment of diseases of the central nervous system, the peripheral nervous system, the cardiovascular system, the pulmonary system, the gastrointestinal system and the endocrinological system.

The compounds of this invention interact with the histamine H₃ receptor and are accordingly particularly useful in the treatment of a variety of diseases or conditions in which histamine H₃ interactions are beneficial.

In one aspect, the invention provides the use of a compound according to formula I, or salts thereof, in a pharmaceutical composition. The pharmaceutical composition may in another aspect of the invention comprise, as an active ingredient, at least one compound according to formula I, or salts thereof, together with one or more pharmaceutically acceptable carriers or excipients. In another aspect, the invention provides such a pharmaceutical composition in unit dosage form, comprising from about 0.05 mg to about 1000 mg, for example, from about 0.1 mg to about 500 mg, such as from about 0.5 mg to about 200 mg, of the compound according to formula I.

In another aspect, the invention provides the use of a compound of this invention for the preparation of a pharmaceutical composition for the treatment of diseases and disorders in which an inhibition of the H₃ histamine receptor has a beneficial effect.

In another aspect, the invention provides the use of a compound of this invention for the preparation of a pharmaceutical composition having histamine H₃ antagonistic activity or histamine H₃ inverse agonistic activity.

In another aspect the invention provides the use of a compound of this invention for the preparation of a pharmaceutical composition for the reduction of weight.

In another aspect, the invention provides the use of a compound of this invention for the preparation of a pharmaceutical composition for the treatment of overweight or obesity.

In another aspect, the invention provides the use of a compound of this invention for the preparation of a pharmaceutical composition for the suppression of appetite or for satiety induction.

In another aspect, the invention provides the use of a compound of this invention for the preparation of a pharmaceutical composition for the prevention and/or treatment of disorders and diseases related to overweight or obesity, such as dyslipidaemia, coronary heart disease, gallbladder disease, osteoarthritis and various types of cancer such as endometrial, breast, prostate and colon cancers.

In another aspect, the invention provides the use of a compound of this invention for the preparation of a pharmaceutical composition for the prevention and/or treatment of eating disorders, such as bulimia or binge eating.

In another aspect, the invention provides the use of a compound of this invention for the preparation of a pharmaceutical composition for the treatment of IGT (Impaired glucose tolerance).

In another aspect, the invention provides the use of a compound of this invention for the preparation of a pharmaceutical composition for the treatment of type 2 diabetes.

In another aspect, the invention provides the use of a compound of this invention for the preparation of a pharmaceutical composition for the delaying or prevention of the progression from IGT to type 2 diabetes.

In another aspect, the invention provides the use of a compound of this invention for the preparation of a pharmaceutical composition for the delaying or prevention of the progression from non-insulin requiring type 2 diabetes to insulin requiring type 2 diabetes.

In another aspect, the invention provides the use of a compound of this invention for the preparation of a pharmaceutical composition for the treatment of diseases and disorders in which a stimulation of the H3 histamine receptor has a beneficial effect.

In another aspect, the invention provides the use of a compound of this invention for the preparation of a pharmaceutical composition having histamine H₃ agonistic activity.

In another aspect, the invention provides the use of a compound of this invention for the preparation of a pharmaceutical composition for the treatment of allergic rhinitis, ulcer or anorexia.

In another aspect, the invention provides the use of a compound of this invention for the preparation of a pharmaceutical composition for the treatment of Alzheimer's disease, narcolepsy, attention deficit disorders or reduced wakefulness, or for the regulation of sleep.

In another aspect, the invention relates to the use of a compound of this invention for the preparation of a pharmaceutical preparation for the treatment of airway disorders, such as asthma, for regulation of gastric acid secretion, or for treatment of diarrhoea.

In another aspect, the invention provides a method for the treatment of disorders or diseases related to the H₃ histamine receptor, the method comprising administering to a subject in need thereof an effective amount of a compound of the general formula I, or salts, solvates, or solvated salts thereof, or of a pharmaceutical composition comprising such a compound.

In another aspect, the invention provides a method as described above, wherein the effective amount of the compound of the general formula I, or salts, solvates, or solvated salts thereof, is in the range of from about 0.05 mg to about 2000 mg, preferably from about 0.1 mg to about 1000 mg, and more preferably from about 0.5 mg to about 500 mg per day.

In one aspect, the invention relates to compounds which exhibit histamine H₃ receptor antagonistic activity or inverse agonistic activity and which may accordingly be useful in the treatment of a wide range of conditions and disorders in which histamine H₃ receptor blockade is beneficial.

In another aspect, the invention provides a method for reduction of weight, the method comprising administering to a subject in need thereof an effective amount of a compound of this invention.

In another aspect, the invention provides a method for treatment of overweight or obesity, the method comprising administering to a subject in need thereof an effective amount of a compound of this invention.

In another aspect, the invention provides a method for suppression of appetite or for satiety induction, the method comprising administering to a subject in need thereof an effective amount of a compound of this invention.

In another aspect, the invention provides a method for prevention and/or treatment of disorders or diseases related to overweight or obesity, such as dyslipidaemia, coronary heart disease, gallbladder disease, osteoarthritis and various types of cancer, for example, endometrial, breast, prostate or colon cancer, the method comprising administering to a subject in need thereof an effective amount of a compound of this invention.

In another aspect, the invention provides a method for prevention and/or treatment of eating disorders, such as bulimia and binge eating, the method comprising administering to a subject in need thereof an effective amount of a compound of this invention.

In another aspect, the invention provides a method for the treatment of IGT (Impaired glucose tolerance), the method comprising administering to a subject in need thereof an effective amount of a compound of this invention.

In another aspect, the invention provides a method for the treatment of type 2 diabetes, the method comprising administering to a subject in need thereof an effective amount of a compound of this invention.

In another aspect, the invention provides a method for the delaying or prevention of the progression from IGT to type 2 diabetes, the method comprising administering to a subject in need thereof an effective amount of a compound of this invention.

In another aspect, the invention provides a method for the delaying or prevention of the progression from non-insulin requiring type 2 diabetes to insulin requiring type 2 diabetes, the method comprising administering to a subject in need thereof an effective amount of a compound of this invention.

In another aspect, the invention relates to compounds which exhibit histamine H₃ receptor agonistic activity and which may accordingly be useful in the treatment of a wide range of conditions and disorders in which histamine H₃ receptor activation is beneficial.

Compounds of this invention may also be used for the treatment of airway disorders (such as asthma), as anti-diarrhoeals, and for the modulation of gastric acid secretion.

Furthermore, compounds of this invention may be used for the treatment of diseases associated with the regulation of sleep and wakefulness, and for the treatment of narcolepsy and attention deficit disorders.

Moreover, the compounds of this invention may be used as CNS stimulants or as sedatives.

The compounds of this invention may also be used for the treatment of conditions associated with epilepsy. Additionally, the compounds of this invention may be used for the treatment of motion sickness and vertigo. Furthermore, they may be useful as regulators of hypothalamo-hypophyseal secretion, as antidepressants, as modulators of cerebral circulation, and in the treatment of irritable bowel syndrome.

Further, compounds of this invention may be used for the treatment of dementia and Alzheimer's disease.

Compounds of this invention may also be useful for the treatment of allergic rhinitis, ulcer or anorexia.

Compounds of this invention may furthermore be useful for the treatment of migraine and for the treatment of myocardial infarction.

In a further aspect of the invention, treatment of a patient with a compound of the present invention is combined with diet and/or exercise.

In a further aspect of the invention, one of more compounds of this invention is/are administered in combination with one or more further active substances in any suitable ratio(s). Such further active agents may, for example, be selected from antiobesity agents, antidiabetics, antidyslipidemic agents, antihypertensive agents, agents for the treatment of complications resulting from or associated with diabetes, and agents for the treatment of complications and disorders resulting from or associated with obesity.

Thus, in a further aspect of the invention one or more compounds of this invention may be administered in combination with one or more antiobesity agents or appetite regulating agents. Such agents may, for example, be selected from the group consisting of CART (cocaine amphetamine regulated transcript) agonists, NPY (neuropeptide Y) antagonists, MC4 (melanocortin 4) agonists, MC3 (melanocortin 3) agonists, orexin antagonists, TNF (tumor necrosis factor) agonists, CRF (corticotropin releasing factor) agonists, CRF BP (corticotropin releasing factor binding protein) antagonists, urocortin agonists, β3 adrenergic agonists such as CL-316243, AJ-9677, GW-0604, LY362884, LY377267 or AZ-40140, MSH (melanocyte-stimulating hormone) agonists, MCH (melanocyte-concentrating hormone) antagonists, CCK (cholecystokinin) agonists, serotonin re-uptake inhibitors such as fluoxetine, seroxat or citalopram, serotonin and noradrenaline re-uptake inhibitors, mixed serotonin and noradrenergic compounds, 5HT (serotonin) agonists, bombesin agonists, galanin antagonists, growth hormone, growth factors such as prolactin or placental lactogen, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, PPAR (peroxisome proliferator-activated receptor) modulators, RXR (retinoid X receptor) modulators, TR β agonists, AGRP (Agouti related protein) inhibitors, opioid antagonists (such as naltrexone), exendin-4, GLP-1 and ciliary neurotrophic factor.

In one embodiment of the invention, an antiobesity agent administered in combination with one or more the compounds of this invention is leptin.

In another embodiment, such an antiobesity agent is dexamphetamine or amphetamine.

In another embodiment, such an antiobesity agent is fenfluramine or dexfenfluramine.

In still another embodiment, such an antiobesity agent is sibutramine.

In a further embodiment, such an antiobesity agent is orlistat.

In another embodiment, such an antiobesity agent is mazindol or phentermine.

In still another embodiment, such an antiobesity agent is phendimetrazine, diethylpropion, fluoxetine, bupropion, topiramate or ecopipam.

In yet a further aspect of the invention, one or more compounds of this invention may be administered in combination with one or more antidiabetic agents. Relevant antidiabetic agents include insulin, insulin analogues and derivatives such as those disclosed in EP 0 792 290, for example, N^{εB29}-tetradecanoyl des (B30) human insulin, EP 0 214 826 and EP 0 705 275, for example, Asp^{B28} human insulin, U.S. Pat. No. 5,504,188, for example, Lys^{B28} Pro^{B29} human insulin, EP 0 368 187, for example, Lantus®, all of which are incorporated herein by reference, GLP-1 derivatives, such as those disclosed in WO 98/08871, incorporated herein by reference, as well as orally active hypoglycaemic agents.

The orally active hypoglycaemic agents preferably comprise imidazolines, sulfonylureas, biguanides, meglitinides, oxadiazolidinediones, thiazolidinediones, insulin sensitizers, α-glucosidase inhibitors, agents acting on the ATP-dependent potassium channel of the β-cells, for example, potassium channel openers such as those disclosed in WO 97/26265, WO 99/03861 and WO 00/37474 which are incorporated herein by reference, or mitiglinide, or a potassium channel blocker, such as BTS-67582, nateglinide, glucagon antagonists, such as one of those disclosed in WO 99/01423 and WO 00/39088, both of which are incorporated herein by reference, GLP-1 agonists, such as those disclosed in WO 00/42026, incorporated herein by reference, DPP-IV (dipeptidyl peptidase-IV) inhibitors, PTPase (protein tyrosine phosphatase) inhibitors, inhibitors of hepatic enzymes involved in stimulation of gluconeogenesis and/or glycogenolysis, glucose uptake modulators, GSK-3 (glycogen synthase kinase-3) inhibitors, compounds modifying the lipid metabolism such as antilipidemic agents, compounds lowering food intake, PPAR (peroxisome proliferator-activated receptor) and RXR (retinoid X receptor) agonists, such as ALRT-268, LG-1268 or LG-1069.

In one embodiment of the invention, one or more compounds of this invention may be administered in combination with insulin or an insulin analogue or derivative, such as N^{εB29}-tetradecanoyl des (B30) human insulin, Asp^{B28} human insulin, Lys^{B28} Pro^{B29} human insulin, Lantus®, or a mix-preparation comprising one or more of these.

In a further embodiment of the invention, one or more compounds of this invention may be administered in combination with a sulfonylurea, for example, tolbutamide, chlorpropamide, tolazamide, glibenclamide, glipizide, glimepiride, glicazide or glyburide.

In another embodiment of the invention, one or more compounds of this invention may be administered in combination with a biguanide, for example, metformin.

In yet another embodiment of the invention, one or more compounds of this invention may be administered in combination with a meglitinide, for example, repaglinide or nateglinide.

In still another embodiment of the invention, one or more compounds of this invention may be administered in combination with a thiazolidinedione insulin sensitizer, for example, troglitazone, ciglitazone, pioglitazone, rosiglitazone, isaglitazone, darglitazone, englitazone, CS-011/CI-1037 or T 174, or a compound disclosed in WO 97/41097, WO 97/41119, WO 97/41120, WO 00/41121 and WO 98/45292, all of which are incorporated herein by reference.

In still another embodiment of the invention, one or more compounds of this invention may be administered in combination with an insulin sensitizer, for example, such as GI 262570, YM-440, MCC-555, JTT-501, AR-H039242, KRP-297, GW-409544, CRE-16336, AR-H049020, LY510929, MBX-102, CLX-0940, GW-501516, or a compound disclosed in WO 99/19313, WO 00/50414, WO 00/63191, WO 00/63192 or WO 00/63193 or in WO 00/23425, WO 00/23415, WO 00/23451, WO 00/23445, WO 00/23417, WO 00/23416, WO 00/63153, WO 00/63196, WO 00/63209, WO 00/63190 or WO 00/63189, all of which are incorporated herein by reference.

In a further embodiment of the invention, one or more compounds of this invention may be administered in combination with an α-glucosidase inhibitor, for example, voglibose, emiglitate, miglitol or acarbose.

In another embodiment of the invention, one or more compounds of this invention may be administered in combination with an agent acting on the ATP-dependent potassium channel of the β-cells, for example, tolbutamide, glibenclamide, glipizide, glicazide, BTS-67582 or repaglinide.

In yet another embodiment of the invention, one or more compounds of this invention may be administered in combination with nateglinide.

In still another embodiment, one or more compounds of this invention may be administered in combination with an antihyperlipidemic agent or antilipidemic agent, for example, cholestyramine, colestipol, clofibrate, gemfibrozil, lovastatin, pravastatin, simvastatin, probucol or dextrothyroxine.

In still another embodiment of the invention, one or more compounds of this invention may be administered in combination with an antilipidemic agent, for example, cholestyramine, colestipol, clofibrate, gemfibrozil, lovastatin, pravastatin, simvastatin, probucol or dextrothyroxine.

In another aspect of the invention, one or more compounds of this invention may be administered in combination with more than one of the above-mentioned compounds, for example, in combination with metformin and a sulfonylurea such as glyburide; a sulfonylurea and acarbose; nateglinide and metformin; acarbose and metformin; a sulfonylurea, metformin and troglitazone; insulin and a sulfonylurea; insulin and metformin; insulin, metformin and a sulfonylurea; insulin and troglitazone; insulin and lovastatin; etc.

Furthermore, one or more compounds of this invention may be administered in combination with one or more antihypertensive agents. Examples of antihypertensive agents are β-blockers such as alprenolol, atenolol, timolol, pindolol, propranolol and metoprolol, ACE (angiotensin converting enzyme) inhibitors such as benazepril, captopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin. Further reference can be made to Remington: The Science and Practice of Pharmacy, 19^{th} Edition, Gennaro, Ed., Mack Publishing Co., Easton, Pa., 1995.

It should be understood that any suitable combination of compounds according to the invention with diet and/or exercise, one or more of the above-mentioned compounds and optionally one or more other active substances are considered to be within the scope of the present invention.

### Pharmaceutical Compositions

The compounds of this invention may be administered alone or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques, such as those disclosed in Remington: The Science and Practice of Pharmacy, 19^{th} Edition, Gennaro, Ed., Mack Publishing Co., Easton, Pa., 1995.

The pharmaceutical compositions may be specifically formulated for administration by any suitable route, such as the oral, rectal, nasal, pulmonary, topical (including buccal and sublingual), transdermal, intracisternal, intraperitoneal, vaginal or parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings, such as enteric coatings, or they can be formulated so as to provide controlled release of the active ingredient, such as sustained or prolonged release according to methods well known in the art.

Liquid dosage forms for oral administration include solutions, emulsions, suspensions, syrups and elixirs.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. Depot injectable formulations are also to be understood as being within the scope of the present invention.

Other suitable administration forms include suppositories, sprays, ointments, cremes, gels, inhalants, dermal patches, implants etc.

A typical oral dosage of a compound claimed herein is in the range of from about 0.001 to about 100 mg/kg body weight per day, preferably from about 0.01 to about 50 mg/kg body weight per day, and more preferably from about 0.05 to about 10 mg/kg body weight per day, administered in one or more doses, such as from 1 to 3 doses. The exact dosage will depend upon the frequency and mode of administration, the sex, age, weight and general condition of the subject treated, the nature and severity of the condition treated and any concomitant diseases to be treated, and other factors evident to those skilled in the art.

The formulations may conveniently be presented in unit dosage form by methods known to those skilled in the art. A typical unit dosage form for oral administration one or more times per day, such as from 1 to 3 times per day, may contain from 0.05 to about 1000 mg, preferably from about 0.1 to about 500 mg, and more preferably from about 0.5 mg to about 200 mg of a compound claimed herein.

For parenteral routes, such as intravenous, intrathecal, intramuscular and similar administration, typical doses are of the order of about half the dose employed for oral administration.

The compounds of this invention are generally utilized as the free substance or as a pharmaceutically acceptable salt thereof. One example is an acid addition salt of a compound having a free base functionality. When a compound of the formula I contains a free base functionality, such salts are prepared in a conventional manner by treating a solution or suspension of the free base form of the compound of formula I with a chemical equivalent (acid-base equivalent) of a pharmaceutically acceptable acid. Representative examples of relevant inorganic and organic acids are mentioned above. Physiologically acceptable salts of a compound of the invention having a hydroxy group include the anion of said compound in combination with a suitable cation, such as sodium or ammonium ion.

For parenteral administration, solutions of the compounds of this invention in sterile aqueous solution, aqueous propylene glycol or sesame or peanut oil may be employed. Such aqueous solutions should be suitably buffered if necessary, and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylenes or water. Similarly, the carrier or diluent may include any sustained release material known in the art, such as glyceryl monostearate or glyceryl distearate, alone or mixed with a wax. The pharmaceutical compositions formed by combining the novel compounds of this invention and the pharmaceutically acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and which may include a suitable excipient. These formulations may be in the form of powder or granules, as a solution or suspension in an aqueous or non-aqueous liquid, or as an oil-in-water or water-in-oil liquid emulsion.

If a solid carrier is used for oral administration, the preparation may be tabletted, placed in a hard gelatine capsule in powder or pellet form or it can be in the form of a troche or lozenge. The amount of solid carrier may vary widely, but will usually be from about 25 mg to about 1 g. If a liquid carrier is used, the preparation may be in the form of a syrup, emulsion, soft gelatine capsule or sterile injectable liquid, such as an aqueous or non-aqueous liquid suspension or solution.

If desired, the pharmaceutical composition of this invention may comprise the compound of the formula I in combination with one or more further pharmacologically active substances, for example, substances chosen among those described in the foregoing.

### Experimental Section

### General

The following abbreviations have been applied: DCM (dichloromethane), DCP (1,2-dichloropropane), DIC (1,3-diisopropylcarbodiimide), DMSO (dimethylsulfoxide), EDAC (1-ethyl-3-(3-dimethylaminopropyl)carbodiimide hydrochloride), EtOAc (ethyl acetate), HOAt (1-hydroxy-7-azabenzotriazole), NMP (N-methylpyrrolidin-2-one), TEA (triethylamine), TFA (trifluoroacetic acid).

Automated parallel synthesis of compound libraries was performed on an Advanced ChemTech 384 HTS synthesizer equipped with four Ares reactors (4×96 wells) and a fast delivery multiprobe.

LC/MS analysis was performed on a LC/UV/ELSD system consisting of a Hewlett Packard series 1100 HPLC system, a Perkin Elmer Series 200 Autosampler a Sedex 75 evaporative light scattering detector and an ES single quadrupole PE-SCIEX API 150EX. The RP-HPLC system was equipped with a Waters XTerra® MS C₁₈ (5 m) 3.0×50 mm column with a flow rate of 1 mL/min. Elution with a gradient of 5 to 90% of solvent B in solvent A within 7.5 min and then 90% solvent B for 0.5 min (solvent A: 0.1% TFA in Milli-Q water; solvent B: 0.1% TFA in CH₃CN).

Automated purification was performed on a Gilson 2.11 MS-12 equipped with a XTerra®Prep MS C₁₈ 100 mm column with flow rate 10 mL/min. Injection volume 0.3 mL DMSO. Elution with a gradient of 10 to 100% of solvent B in solvent A within 11 min and then 100% solvent B for 4 min (solvent A: 0.1% TFA in Milli-Q water; solvent B: 0.1% TFA in CH₃CN). Samples were collected in 24-well plates and evaporated in a GeneVac HT12. After fraction collection, the preparative chromatograms were processed by in-house software and the amount of purified compound in each fraction was calculated from the integrated ELSD response that was matched up to an external standard (Fmoc-Phe-OH, 10 mg/ml). No further quantification was performed.

Melting points were determined in open capillary tubes on a Büchi 535 melting point apparatus and are uncorrected. The structures of re-synthesised compounds are consistent with spectroscopic data and satisfactory elemental analyses were obtained within ±0.4% of theoretical values unless otherwise noted. ¹H NMR spectra were recorded on a Bruker DRX300 or Bruker WM400 spectrometer with TMS as internal standard, with chemical shifts quoted in ppm (δ) in the solvents indicated.

### [³⁵S]GTPγS Assays

CHO-cells, stably expressing the human histamine H₃ receptor, were harvested and membranes were prepared by threefold homogenization in a HEPES buffer (20 mM HEPES, 0.1-10 mM EDTA; pH 7.4). The antagonist potency of a compound was measured as its ability to inhibit the binding of [³⁵S]GTPγS to H₃ receptor membranes in the presence of 10 nM (R)-α-methylhistamine (RAMHA). Test compounds was diluted in assay buffer (20 mM HEPES, 120 mM NaCl, 10 mM MgCl₂, pH 7.4) followed by addition of 10 nM RAMHA, 3 μM GDP, 2.5 μg membranes, 0.5 mg SPA beads, and 0.1 nM [³⁵S]GTPγS. The test compounds were first tested at a fixed concentration (1.4 μM or 3.5 μM) and the potencies were expressed as % inhibition. The hits were subsequently tested in full dose-response curves for functional IC₅₀ determinations. After 2 h incubation at RT with gentle shaking of the plate, the plate was centrifuged at 370 RCF for 10 min and subsequently the radioactivity was counted in a Packard TopCount-NXT.

### Data Analysis

The IC₅₀ values were calculated by non-linear regression analysis using GraphPad Prism. The functional Kᵢ values were calculated from the IC₅₀ values using the Cheng-Prusoff relationship: Kᵢ=IC₅₀/(1+(S/EC₅₀)) where S represents the concentration of RAMHA used (10 nM) and EC₅₀ represents the mean EC₅₀-value (±SEM) of RAMHA in more than 60 separate agonist [³⁵S]GTPγS binding experiments (i.e. 2.7±0.15 nM).

### Generation of a Pharmacophore Model for Compound 1

A conformational analysis was performed in Maestro version 7 using the MMFF force field, water as the solvation model and the MCMM method for the conformational search. The conformational search was run with 2000 steps. The global energy minimum was used to map pharmacophore features in Catalyst version 4.6 to compound 1. The hydrophobic and positive ionisable features were mapped with the default tolerance values (1.5 Å). A Catalyst database from the virtual scope of library 2 (518 compounds) was built with the fast generation method and with a maximum of 100 conformations. The database was searched with the Fast Flexible Search method to give 384 compounds that were selected for synthesis as library 2.

### Automated Parallel Synthesis of Amide Library 2

The TFP resin 4 (0.7 mmol/g, 65 mg, 0.045 mmol) was manually added to each of the 384 wells of an Advanced ChemTech 384 HTS synthesiser. According to the library design each well was treated individually with 37 different carboxylic acids (0.36 mmol) in NMP/DCP (1:1) (1.5 mL) followed by DIC (0.028 mL, 0.18 mmol) in NMP (0.1 mL). The array was then shaken for 16 h. The solvents were removed and the acylated resin 5 was washed with NMP (4×1 mL) and DCM (10×1 mL).

According to the library design each well was treated individually with 14 different amines (0.041 mmol) in DCP (1 mL) followed by TEA (8.2 mg, 0.081 mmol) in DCP (0.1 mL). The array was shaken for 20 h and then the reactors were emptied into glass vials. Each reactor well was treated with CH₃CN (1 mL) and shaken for 90 min to rinse the resin for product. The washing was emptied into the same set of 4 mL glass vials which were then evaporated to dryness. The residues were dissolved by addition of DMSO (0.25 mL) to each vial. A random subset of 40 samples were analysed by LC-MS showing that the desired compound was present in 83% of the samples with a purity >50%.

The average yield of the library was estimated to 50% and the 384 samples were diluted with DMSO (10×) and finally with H₂O to a final screening concentration of 1.4 μM. The diluted samples were then tested in the automated [³⁵S]GTPγS inhibition assay and the results are shown in Table 1.

### Automated Parallel Synthesis of Amide Library 3

A suspension of TFP resin 4 (Argotech 1.1 mmol/g, 41 mg, 0.045 mmol) in NMP/DCM (1:1) was added to each of the 357 wells of an ACT 384 synthesiser. The solvents were removed and the resin was washed with DCM (1 mL/well). According to the library design each well was treated individually with 119 different carboxylic acids (0.36 mmol) in NMP/DCM (1:1) (1.5 mL) followed by DIC (0.028 mL) in NMP (0.1 mL). The array was then shaken for 16 h. The solvents were removed and the acylated resin 5 was washed with NMP (4×1 mL) and DCM (10×1 mL).

According to the library design each well was treated individually with 3 different amines (0.041 mmol) in DCP (1 mL) followed by TEA (0.081 mmol) in DCP (0.1 mL). The array was shaken for 20 h and then emptied into 2 mL deepwell plates. Each well was treated with CH₃CN (1 mL) and left for 2 h to rinse the resin for product. The washing was emptied into the same deepwell plates which were then evaporated 8 h at RT and 4 h at 40° C. The residues were dissolved by addition of DMSO (0.25 mL) to each well.

The 357 samples were submitted for automated LC-MS purification and quantification which recovered 298 samples. The samples were diluted with DMSO until a final concentration of 560 mM for each sample. Finally, the samples were diluted with H₂O to a screening concentration of 3.5 μM. The diluted samples were then tested in the automated [³⁵S]GTPγS inhibition assay and the results are shown in Table 2.

Preparation of Selected Compounds

### 2-(9H-Fluoren-9-yl)-1-(4-(3-(piperidin-1-yl)propyl)piperazin-1-yl)ethanone, dihydrochloride (A18B12)

A mixture of fluoren-9-acetic acid (0.67 g, 3.0 mmol), HOAt (0.42 g, 3.0 mmol), EDAC (0.58 mg, 3.0 mmol) and DCM (20 mL) was stirred at RT for 30 min. 1-(3-Piperidinopropyl)piperazine (0.50 g, 2.4 mmol) was added and the mixture was stirred at RT for 2 days. DCM (25 mL) was added and the mixture was washed with H₂O (3×25 mL). The organic phase was evaporated to dryness and then dissolved in a mixture of 1 N HCl (10 mL) and H₂O (75 mL). The aqueous solution was washed with diethyl ether (2×50 mL), made alkaline with a 4 N NaOH solution and extracted with EtOAc (2×50 mL). The combined organic extracts were dried (MgSO₄) and the solvent was evaporated to give an oily residue. A 0.2 N HCl solution was added until dissolution and then the volatiles were evaporated. The residue was re-evaporated with CH₃CN to give a solid which was stirred with a small portion of CH₃CN. The solid was isolated and dried to give 0.75 g (65%) of A18B12. Mp. 263-265° C. ¹H NMR (400 MHz, DMSO-d₆) δ 1.30-1.46 (m, 1H), 1.65-1.88 (m, 5H), 2.15-2.28 (m, 2H), 2.78-3.63 (m, 16H), 3.99-4.10 (m, 1H), 4.43 (t, J=7.5 Hz, 1H), 4.56-4.69 (m, 1H), 7.29-7.34 (m, 2H), 7.39 (t, J=7.5 Hz, 2H), 7.59-7.61 (m, 2H), 7.88 (d, J=7.5 Hz, 2H), 10.6 (brs, 1H), 11.5 (brs, 1H). MA (C₂₇H₃₆N₃O, 2HCl, H₂O) C, H, N; C: Calc: 63.77; Found: 63.28.

### Benzofuran-2-yl-(4-(3-(piperidin-1-yl)propyl)piperazin-1-yl)methanone, dihydrochloride (A12B12)

A mixture of benzofuran-2-carboxylic acid (0.33 g, 2.0 mmol), HOAt (0.27 g, 2.0 mmol), EDAC (0.38 mg, 2.0 mmol) and DCM (20 mL) was stirred at RT for 30 min. 1-(3-Piperidinopropyl)piperazine (0.38 g, 1.8 mmol) was added and the mixture was stirred at RT for 16 h. DCM (50 mL) was added and the mixture was washed with H₂O (3×30 mL). The organic phase was dried (MgSO₄) and the solvent was evaporated. To the residue was added a 0.2 N HCl solution (30 mL) until dissolution and then the volatiles were evaporated. The residue was re-evaporated with EtOH to give a solid which was stirred with CH₃CN (10 mL). The solid was isolated and dried to give 0.62 g (80%) of A12B12. Mp. 253-255° C. ¹H NMR (300 MHz, DMSO-d₆) δ 1.31-1.47 (m, 1H), 1.63-1.89 (m, 5H), 2.16-2.33 (m, 2H), 2.77-3.66 (m, 14H), 4.46-4.63 (m, 2H), 7.36 (t, J=7.5 Hz, 1H), 7.49 (t, J=7.5 Hz, 1H), 7.54 (s, 1H), 7.69 (d, J=7.5 Hz, 1H), 7.78 (d, J=7.5 Hz, 1H), 10.6 (brs, 1H), 11.6 (brs, 1H). MA (C₂₁H₂₆N₃O₂, 2HCl, 1.5H₂O) C, H, N.

1. A compound of Formula (I): 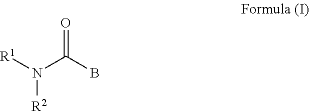
wherein
R¹ and R² are taken together to form a 3-(piperidin-1-yl)propyl)piperazin-1-yl group; and
B is selected from the group consisting of —(CH₂)ₚ-aryl, -alkenylene-aryl, —(CH₂)ₚ-heteroaryl, -alkenylene-heteroaryl, —(CH₂)_{q}—C(O)—(CH₂)ₚ-aryl, and —(CH₂)_{q}—C(O)—(CH₂)ₚ-heteroaryl, —(CH₂)ₚ-fused cycloalkylaryl and —(CH₂)_{q}—C(O)—(CH₂)ₚ-fused cycloalkylaryl wherein the aryl and heteroaryl groups each independently are substituted with from zero to four R^{g}, wherein
p is an integer from 0 to four;
q is an integer from 0 to four; and
each R^{g} independently is selected from the group consisting of hydrogen, —O—Ar¹, Ar¹, alkyl, alkoxy, hydroxyl, halogen, haloalkyl and alkoxyalkyl, wherein the Ar¹ is substituted with from zero to four R^{h}, wherein
Ar¹ is aryl or heteroaryl;
R^{h} is selected from the group consisting of hydrogen, alkyl, alkoxy, hydroxyl, halogen, haloalkyl and alkoxyalkyl;
or a pharmaceutically acceptable salt thereof.

2-7. (canceled)

8. A method of treating a disease mediated by the modulation of the H₃ receptor, the method comprising administering to a subject a compound of Formula (I): 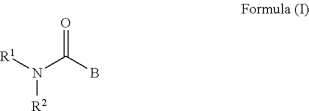
wherein
R¹ and R² are taken together to form a 3-(piperidin-1-yl)propyl)piperazin-1-yl group; and
B is selected from the group consisting of —(CH₂)ₚ-aryl, -alkenylene-aryl, —(CH₂)ₚ-heteroaryl, -alkenylene-heteroaryl, —(CH₂)_{q}—C(O)—(CH₂)ₚ-aryl, and —(CH₂)_{q}—C(O)—(CH₂)ₚ-heteroaryl, —(CH₂)ₚ-fused cycloalkylaryl and —(CH₂)_{q}—C(O)—(CH₂)ₚ-fused cycloalkylaryl wherein the aryl and heteroaryl groups each independently are substituted with from zero to four R^{g}, wherein
p is an integer from 0 to four;
q is an integer from 0 to four; and
each R^{g} independently is selected from the group consisting of hydrogen, —O—Ar¹, Ar¹, alkyl, alkoxy, hydroxyl, halogen, haloalkyl and alkoxyalkyl, wherein the Ar¹ is substituted with from zero to four R^{h}, wherein
Ar¹ is aryl or heteroaryl;
R^{h} is selected from the group consisting of hydrogen, alkyl, alkoxy, hydroxyl, halogen, haloalkyl and alkoxyalkyl;
or a pharmaceutically acceptable salt thereof.

9. (canceled)

10. A pharmaceutical composition comprising: a compound of Formula (I): 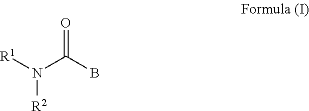
or a pharmaceutically acceptable salt thereof,
wherein
R¹ and R² are taken together to form a 3-(piperidin-1-yl)propyl)piperazin-1-yl group; and
B is selected from the group consisting of —(CH₂)ₚ-aryl, -alkenylene-aryl, —(CH₂)ₚ-heteroaryl, -alkenylene-heteroaryl, —(CH₂)_{q}—C(O)—(CH₂)ₚ-aryl, and —(CH₂)_{q}—C(O)—(CH₂)ₚ-heteroaryl, —(CH₂)ₚ-fused cycloalkylaryl and —(CH₂)_{q}—C(O)—(CH₂)ₚ-fused cycloalkylaryl wherein the aryl and heteroaryl groups each independently are substituted with from zero to four R^{g}, wherein
p is an integer from 0 to four;
q is an integer from 0 to four; and
each R^{g} independently is selected from the group consisting of hydrogen, —O—Ar¹, Ar¹, alkyl, alkoxy, hydroxyl, halogen, haloalkyl and alkoxyalkyl, wherein the Ar¹ is substituted with from zero to four R^{h}, wherein
Ar¹ is aryl or heteroaryl;
R^{h} is selected from the group consisting of hydrogen, alkyl, alkoxy, hydroxyl, halogen, haloalkyl and alkoxyalkyl; and
a pharmaceutically acceptable carrier or diluent.
